(19) United States
(12) Patent Application Publication
(10) Pub. No.: US 2026/0294986 A1
(43) Pub. Date: Oct. 1, 2026

(54) ENCAPSULATION MATERIALS AND METHODS FOR IMPROVED DRUG DELIVERY

(71) Applicant: Government of the United States as represented by the Secretary of the Air Force, Wright-Patterson AFB, OH (US)

(72) Inventors: Margaret C. Warner, Charleston, WV (US); Jie Xu, Atlanta, GA (US)

(21) Appl. No.: 19/632,822

(22) Filed: Mar. 30, 2026

Related U.S. Application Data
(60) Provisional application No. 63/780,789, filed on Mar. 31, 2025.

(51) Int. Cl. A61K 35/747; A61K 35/00; A61K 35/742; C12N 1/20

(52) U.S. Cl. CPC A61K 35/747; A61K 35/742; C12N 1/20; A61K 2035/115

(57) ABSTRACT

A method of creating a spore coated probiotic is provided. The method comprises preparing a spore suspension, extruding that spore suspension. Then incubating the spore extrusion with a non-spore forming probiotic. And, exposing uncoated cells to a heat treatment to facilitate removal.

### CROSS-REFERENCE TO RELATED APPLICATIONS

This application claims the benefit of and priority to U.S. Provisional Application Ser. No. 63/780,789, entitled “Encapsulation Materials and Methods for Improved Drug Delivery,” filed on Mar. 31, 2025, the entirety of which is incorporated by reference herein.

### RIGHTS OF THE GOVERNMENT

The invention described herein may be manufactured and used by or for the Government of the United States for all governmental purposes without the payment of any royalty.

### BACKGROUND OF THE INVENTION

### Field of the Invention

The present invention relates generally to encapsulation materials and methods for improved drug delivery. Particularly, embodiments of the present invention relate to single-cell bacterial encapsulation technology that employs a mechanically extruded spore coat nanoparticle (SCN) as a protective layer.

### DESCRIPTION OF THE RELATED ART

Probiotics are live microorganisms that provide numerous health benefits when consumed in sufficient amounts. They help balance the gut microbiome by promoting the growth of beneficial bacteria while suppressing harmful ones, thereby improving gastrointestinal health and reducing the risk of infections. Probiotics also support the immune system through chemical signaling and by enhancing the body's natural defenses. As a potential alternative to antibiotics, probiotics can reduce reliance on broad-spectrum treatments, thereby mitigating the development of antibiotic resistance. Additionally, they alleviate digestive issues such as diarrhea, irritable bowel syndrome (IBS), and lactose intolerance while improving nutrient absorption. These properties position probiotics as a promising tool for maintaining overall health and combating bacterial infections effectively.

Because probiotics are not typically classified alongside pharmaceuticals used to treat diseases, the FDA does not require their approval before they enter the market. Furthermore, there are no regulations regarding the standardization of probiotic consumption, cell count methods, or preparation requirements. There is also no federal or international mandate to assess the survival rate of probiotic cells after digestion. Mechanical stress and dehydration during the freeze-drying process often damage cell membranes, leading to cell death. Membrane lipid oxidation during storage further contributes to cell mortality. In many commercial probiotics, viable cell counts are already lower than manufacturer claims even before digestion. Simulated digestion studies show that cell counts can drop by one to two log-fold within just five to ten minutes, with some freeze-dried cell capsules showing no viable cells after digestion.

While there is no universally accepted standard for effective probiotic cell counts, beneficial outcomes generally require between 10⁶ and 10⁸ CFU/g. Depending on pre-processing methods, probiotic survival after digestion can range from 0 to 10⁸ CFU/mL, but dried cells consistently show lower viability compared to liquid probiotics. During digestion, cells face challenges such as low pH, mechanical stress, and bile exposure, which degrade cell membranes.

As researchers uncover more about how probiotics interact with and combat pathogenic bacteria in vivo, the next challenge lies in ensuring proper application and consumption. To use probiotics effectively as a primary defense against bacterial infections, they must colonize abundantly and maintain stable populations. Producers must prioritize high cell viability not only after manufacturing but also following oral delivery and digestion. Single-cell encapsulation of probiotics using biologically sourced, highly resistant materials offers a promising solution by equipping beneficial bacteria with the “armor” needed to survive harsh environments within the host body and populate regions requiring microbiome rejuvenation.

Common techniques for single-cell probiotic protection include hydrogel encapsulation, synthetic nano- and microparticles, nanofilm development, and surface functionalization. Techniques like nanofilm development via layer-by-layer deposition and surface functionalization, which leverage engineered surface-expressed proteins or membrane chemistry modifications, demonstrate higher mucoadhesion in the gastrointestinal tract and offer superior protection. However, these methods are still in their infancy and have yet to be optimized for industrial-scale production. Encapsulation methods that protect multiple cells simultaneously, such as hydrogels and microparticles, tend to result in limited colonization and pose challenges in controlling particle size. However, these synthetic materials are widely available for industrial use and often provide better protection and resistance properties due to their non-biological origin.

Accordingly, there is a need in the art to better encapsulate and protect cells.

### SUMMARY OF THE INVENTION

Drawing inspiration from the high resilience of bacterial spores under extreme conditions, methodologies in some embodiments of the invention use a spore coat as a single-cell encapsulation material, providing protections previously only available to cells capable of forming their own endospores. Bacterial spores are known to withstand various stresses, largely due to the thickness and structure of their multilayered spore coat, composed of over 70 different proteins. It has been demonstrated that lipid-protein nanoparticles derived from spore coats can be extracted and attached to vegetative cells of the same genus, giving these cells advantages over spores. Methodologies in some embodiments of the invention show that spore coat nanoparticle (SCN) can also be aggregated onto a surface of non-spore-forming, unrelated, Gram-positive bacteria, providing them with spore-like protection. Moreover, SCN incubation and attachment confer resistance to high thermal extremes and extreme pH-traits previously unknown in these cells. These findings contribute to advancements in biologically-based materials for cellular encapsulation and protection.

These and other aspects of the present invention are described in the Detailed Description of the Invention below and the accompanying figures. Other aspects and features of embodiments of the present invention will become apparent to those of ordinary skill in the art upon reviewing the following description of specific, exemplary embodiments of the present invention in concert with the figures. While features of the present invention may be discussed relative to certain embodiments and figures, all embodiments of the present invention can include one or more of the features discussed herein. Further, while one or more embodiments may be discussed as having certain advantageous features, one or more of such features may also be used with the various embodiments of the invention discussed herein. In similar fashion, while exemplary embodiments may be discussed below as device, system, or method embodiments, it is to be understood that such exemplary embodiments can be implemented in various devices, systems, and methods of the present invention.

### BRIEF DESCRIPTION OF THE DRAWINGS

The accompanying drawings, which are incorporated in and constitute a part of this specification, illustrate embodiments of the invention and, together with a general description of the invention given above, and the detailed description given below, serve to explain the invention.

FIG. 1A shows characterization of SCN encapsulated probiotics LC and LA via SEM images of LC@168;

FIG. 1B shows characterization of SCN encapsulated probiotics LC and LA via SEM images of LC@6051;

FIG. 1C shows characterization of SCN encapsulated probiotics LC and LA via SEM images of LC@GS (likely with salt accumulation from media;

FIG. 1D shows SEM images of LA coated in all three (168/6051/GS) SCN;

FIG. 2A shows survival of SCN encapsulated probiotics under 85° C. heat treatments for 10 and 75 minutes;

FIG. 2B shows survival of SCN encapsulated probiotics under GFS-pepsin exposure for two hours;

FIG. 2C shows a comparison of the survival rates under each independent treatment type;

FIG. 3A shows characterizations of spore coated nanoparticles (SCN) by scanning electron microscopy in accordance with embodiments of the invention;

FIG. 3B shows zeta size average of G. stearothermophilus produced SCN in accordance with embodiments of the invention;

FIG. 3C shows zeta size average of B. subtilis 6051 produced SCN in accordance with embodiments of the invention;

FIG. 4 shows the zeta potential of SCN in accordance with embodiments of the invention;

FIG. 5A shows scanning electron microscopy images of cross-species coated cells of spore-forming species including 168@6051 after 6 hours in accordance with embodiments of the invention;

FIG. 5B shows scanning electron microscopy images of cross-species coated cells of spore-forming species more sparsely coated 168@GS in accordance with embodiments of the invention;

FIG. 5C shows scanning electron microscopy images of cross-species coated cells of spore-forming species including uncoated, vegetative 168 cell in accordance with embodiments of the invention;

FIG. 6A shows the results of cross-species coated vegetative B. subtilis cell counts (6051 and 6051@168) with p=0.006 when treated at 95° C. for 10 minutes in accordance with embodiments of the invention;

FIG. 6B shows the results of cross-species coated vegetative B. subtilis cell counts (6051 and 6051@GS) with p=0.006 when treated at 95° C. for 10 minutes in accordance with embodiments of the invention;

FIG. 6C shows the results of cross-species coated vegetative B. subtilis cell counts (168 and 168@GS) with p<0.001 when treated at 95° C. for 10 minutes in accordance with embodiments of the invention;

FIG. 7 shows survival of SCN encapsulated probiotics under 85° C. heat treatments for 10 and 75 minutes in accordance with embodiments of the invention;

FIG. 8 shows survival of SCN encapsulated probiotics under GFS-pepsin exposure for 2 hours in accordance with embodiments of the invention; and

FIG. 9 shows total growth of S. enteritis on selective media after co-culturing with uncoated L. acidophilus and L. casei, 168/6051/and GS-coated L. acidophilus and L. casei, as well as S. entiritis grown alone as a control in accordance with embodiments of the invention.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION OF THE INVENTION

### Bacterial Spores

Multiple genera of bacteria are capable of transitioning between actively growing and metabolically-static, defensive states. Vegetative cells refer to the part of the population that are actively undergoing metabolism and cell division, carrying out a relatively regular length life cycle for non-extremophilic bacteria. Bacterial endospores, from here on out referred to as spores, are dormant, highly resistant cells formed by a few genera of Gram-positive bacteria. They form a specialized, multilayer spore coat made up of over 70 different proteins interlaced in a lipid membrane matrix which allows them to withstand a variety of extreme environmental attacks and disturbances. Primarily, cells of the family Bacillaceae and Clostridiaceae are capable of forming spores, with a few others. Spore-forming cells, by majority, include the largely aerobic genus Bacillus and the anaerobic genus Clostridia.

Some of the of the illustrated embodiments in this disclosure focus on members of Bacillaceae, as they tend to be non-pathogenic. The general make-up of a Bacillus sp. spore coat is very well-studied and includes the crust, outer coat, inner coat, basement layer, outer forespore membrane, cortex, and inner forespore membrane, all encompassing the core which contains the chromosome. This core has been partially dehydrated and water is replaced by calcium dipicolinic acid (CaDPA), which helps protect the DNA from heat denaturation and UV damage. In terms of triggering spore coat formation and attachment to the outside of the cell membrane, SpoIVA, SpoVM, SpoVID, SafA, and CotE are the morphogenetic proteins identified as having the most direct effect on every layer of coat assembly in sporulation. Specific to note, SpoIVA, SpoVM, and SpoVID play roles in the basement layer assembly, with SafA directing the inner coat aggregation, CotE on the outer coat, and proteins CotX, CotY, and CotZ functioning in the development of the crust.

### Process of Sporulation

A cell or population undergoes sporulation due to a series of gene expression activating critical proteins. These genetic changes are triggered by the lack of sufficient key nutrients or moisture in the cells' environment, like carbon, nitrogen, and phosphate. Once a cell undergoes sporulation, the vegetative cell divides asymmetrically, producing a mother cell and a forespore; the latter is engulfed by the mother and continues development in a separate compartment within the mother. While in this separated region, the forespore becomes dehydrated and the spore coat begins to develop. Once fully formed, the mother cell lyses and leaves the spore to wait until more favorable environmental conditions are present. The complete process of sporulation can be triggered and carried out in as few as five hours, but typically takes between seven and ten hours. Once a cell has undergone sporulation, it can remain viable for hundreds to thousands of years, and is able to produce a vegetative population following germination. There are even some spores that have been revived after preservation for between 25-40 million years, suggesting that the environment the cell sporulates in may contribute to its survival over an extremely long period of time.

The exposure of spores to specific nutrients, which includes amino acids, sugars, and inorganic salts, lead cells to undergo germination. These germinants must reach receptors on the cell, meaning they will pass through multiple layers of the spore coat. Though the outer layers of the spore coat have higher permeability, the inner membrane is much less permeable. The cell will take in water and break apart its spore coat, of which the inner forespore membrane becomes the new cellular membrane. The process of germination can occur in as little as a few minutes, but usually takes 15 or more minutes. Germination allows for the spore to perceive and respond to a change in its environment, then begin growth quickly after. The receptors on the inner spore membrane will receive nutrient signals from the environment, causing a series of protein receptor complexes to change conformation and alter permeability for potassium, hydrogen, and sodium across the spore coat. This leads to rehydration of the spore core and degradation of the spore coat, producing an actively growing vegetative cell. A germinated cell can withstand normal environmental fluctuations, but can be triggered into sporulation if necessary. Otherwise, a germinated cell will continue on with cell division and a regular bacterial life cycle.

### Sporulation and Maturation

In an illustrative embodiment, Bacillus subtilis 168 M (ATCC #23857), Bacillus subtilis 6051 (ATCC #6051-U), and Geobacillus stearothermophilus (STERIS #NA030) bacterial stocks were streaked onto Luria Broth (LB) plates and incubated aerobically for 24 hours at 37° C. Single colonies were selected to inoculate 50 mL of LB and were grown aerobically for 24 hours at 37° C. and 150 rpm. The 50 mL LB conical tubes were centrifuged at 4500×g for 10 minutes to obtain vegetative cell pellets, then the supernatant was removed. The cell pellet was resuspended in 1 mL Phosphate Buffered Saline (PBS). The PBS suspensions were used to inoculate pre-made Spordex Culture Media (STERIS Life Sciences, #NA114). These Spordex cultures were incubated for 24-48 hours (until significant color change) at 37° C. and 150 rpm. After incubation, the cultures matured at 4° C. for at least four days, then were heated to 95° C. for 10 minutes to remove all remaining vegetative cells.

A 15 mL HistoDenz solution (50% w/v) (Millipore Sigma, #D2158) in a 50 mL conical tube was brought to room temperature. Heat-treated Spordex culture was carefully layered on top of the solution. The tubes were then centrifuged at 4,500×g for 10 minutes. After centrifugation, the top three layers (upper clear layer, dense opaque layer, and cloudy layer) were removed, leaving the last few millimeters of the cloudy layer and the pellet undisturbed. The pellet was resuspended in the remaining liquid using a pipette and mixed by vortexing for approximately 30 seconds. The mixture was transferred to a 1.5 mL microcentrifuge tube and centrifuged at 16,000×g for 5 minutes. The supernatant was discarded, and the pellet was resuspended in 1 mL of cold PBS, followed by vortexing for about 30 seconds. The microcentrifuge tube was centrifuged again at 16,000×g for 2 minutes. The supernatant was removed, and the pellet was resuspended in 1 mL of cold PBS. This wash step (centrifugation and resuspension) was repeated two more times for a total of three washes. After the third wash, the tube was centrifuged one final time at 16,000×g for 2 minutes. The supernatant was removed, and the pellet was resuspended in 0.5 mL of cold PBS. The resulting spore suspension was stored at 4° C. for at least 10 days to allow for maturation.

After purified spores had matured at 4° C. for at least 10 days, 1 mL of spore suspension was used to extrude spore coat nanoparticles. An 800 nm polycarbonate filter was inserted in the hand-held mini lipid extruder (Avanti Polar Lipids), the extruder was set up according to its instructions, and the spore suspension was extruded back and forth 13 times. Next, the 800 nm filter was replaced with a 400 nm filter and the filter supports were removed and replaced with new ones. The spore suspension was extruded back and forth another 13 times. The twice filtered solution was deposited into a clean microcentrifuge tube. Next, to remove all cell debris and any complete spores, the solution was centrifuged at 2,000×g for 5 minutes. The supernatant was kept and the pellet discarded. The supernatant was centrifuged at 12,000×g for 10 minutes to obtain a pellet of spore coat particles. The supernatant of this centrifugation was then removed. The pellet was resuspended in cold PBS and mixed by vortexing for 30 seconds. The previous 2 steps were repeated twice more, with a final suspension of the SCN pellet in 300 μL of deionized water. The remaining solution contained spore coat nanoparticles, which could be used immediately to coat bacterial cells.

Lactobacillus acidophilus (ATCC #BAA-2832) (referred to as LA) and Lactobacillus casei (ATCC #393) (referred to as LC) were selected for this illustrated embodiment due to their far-removed relationship from the spore forming species used to create the SCN, as well as their use as successful probiotics in reducing pathogenic bacteria yields in chickens. Primarily, these species differ by regeneration time, where LA proliferation time takes about 75 minutes and LC proliferation time is around 130 minutes. Additionally, LA is microaerobic and prefers anaerobic growth conditions whereas LC is a facultative anaerobe. After a 24 hour incubation at 37° C. and 150 rpm, the cells are centrifuged and resuspended in PBS, then treated at 95° C. for ten minutes to remove any uncoated cells from the culture.

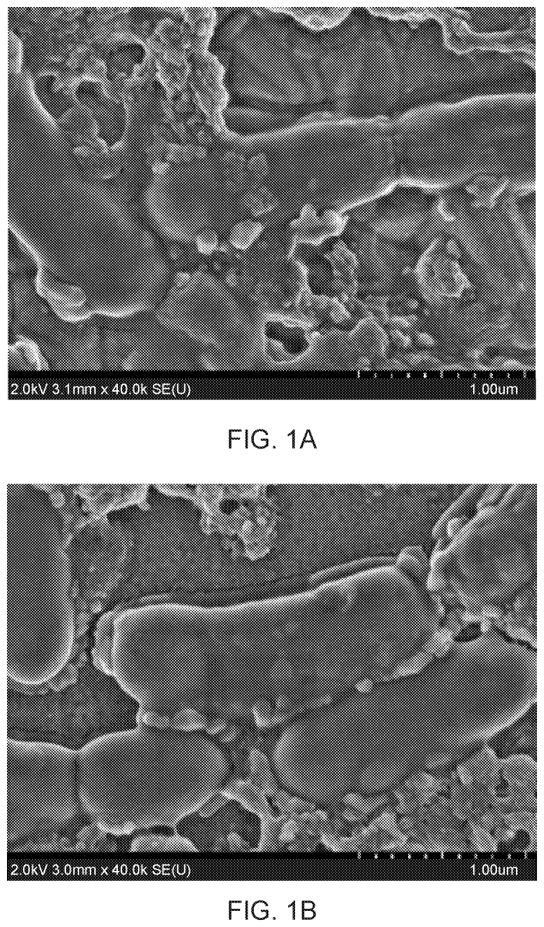

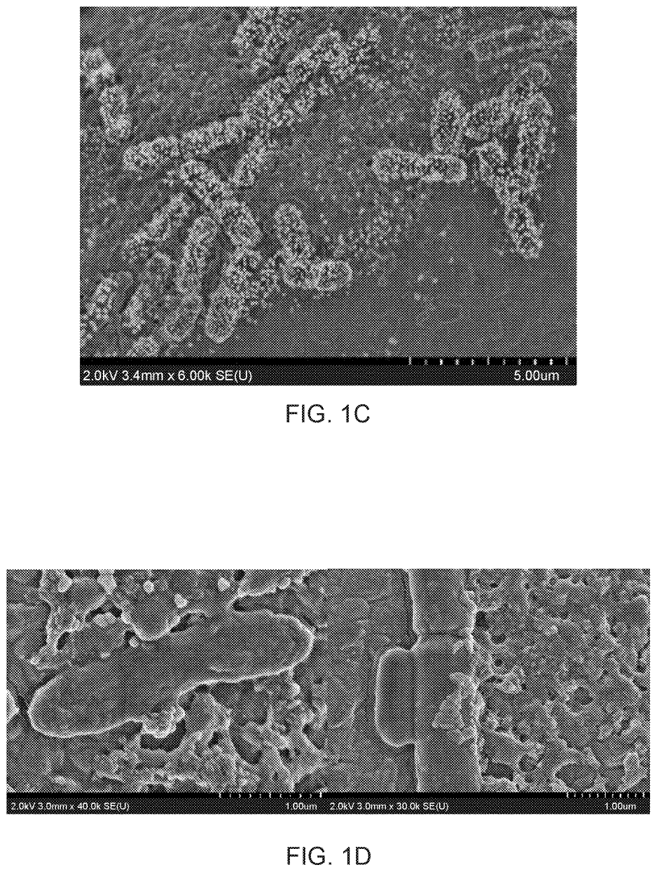

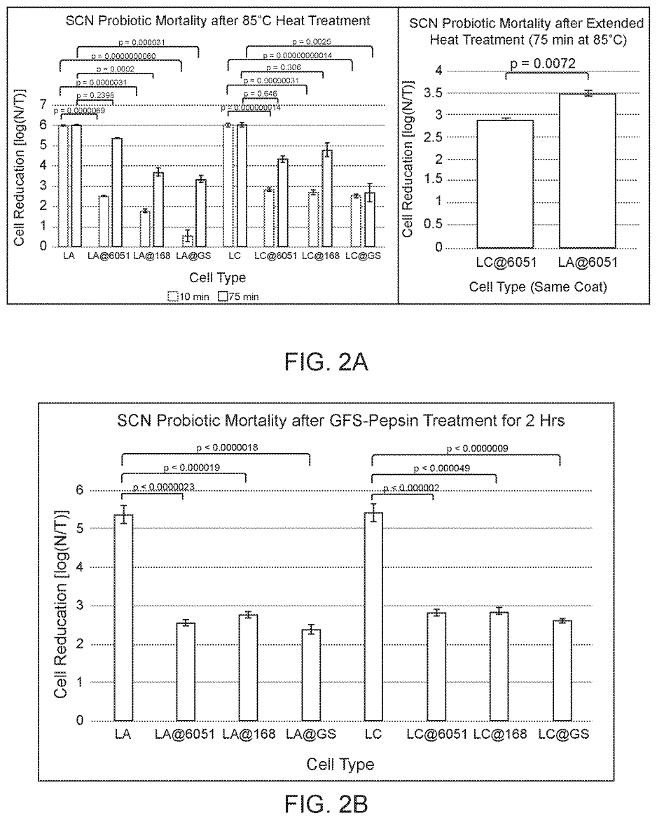

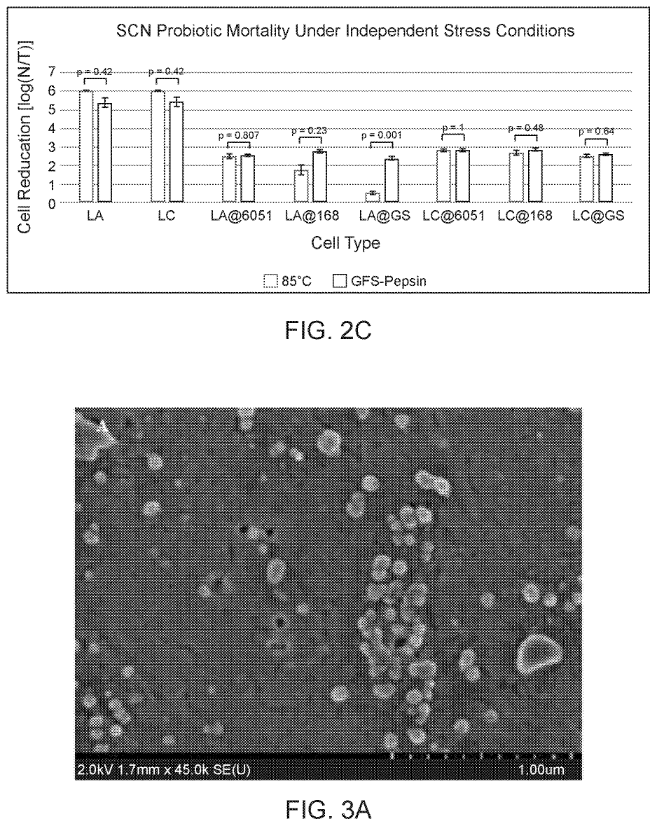

Carrying out the same protocols for SCN formation and purification above, the pure spore coat extrusions were then incubated with overnight cultures of the non-spore forming probiotics LA and LC. After a 24 hour incubation at 37° C. and 150 rpm, the cells are centrifuged and resuspended in PBS, then treated at 95° C. for ten minutes to remove any uncoated cells from the culture. The same staining and SEM image analysis are used to verify cell coating as with cross-species coated cells as seen in the SEM images in FIGS. 1A-1D. Overnight cultures matched to the optical density of coated cells are used as controls in determining resistance characteristics of the probiotics. Uncoated cells exposed to heat treatment at 85° C. for ten minutes or 75 minutes have zero survival and uncoated cells exposed to Gastric Fluid Simulant (GFS)-pepsin for the standard two hour treatment have less than ten cells survive total as illustrated in FIGS. 2A-2C. FIG. 2A shows survival of SCN encapsulated probiotics under 85° C. heat treatments for 10 and 75 minutes (ANOVA for 10 minutes: p-value=0.000000000149; ANOVA for 75 minutes: p-value=0.0000187; Dunnett's test: p-value <0.05 shown in black; N=3) and FIG. 2B is under GFS-pepsin exposure for two hours (ANOVA: p-value=0.0000000044; one-tailed t-test: p-value <0.05 shown in black; N=3). FIG. 2C illustrates a comparison of the survival rates under each independent treatment type (two-tailed t-test: almost all p-values >0.05, no statistical difference within cell types; N=3). Colony forming units (CFUs) are graphed as a log reduction, where N=total number of cells estimated from original OD value and T=number of cells after treatment. Error bars indicate one standard deviation calculated from the log transformed data, graphed as

$${σ}_{N / T} = \sqrt{{σ}_{N}^{2} + {σ}_{T}^{2}} . \qquad ( 1 )$$

Further in this illustrated embodiment, in 1.5 mL microcentrifuge tubes, using three independent replicates, 100 μL of an overnight incubation of bacterial cell of interest, typically normalized to 0.6-0.8 OD600, and 300 μL of the spore coat nanoparticle suspension was added, plus an additional 500 μL of fresh LB. The cells were incubated for 6-24 hours at 37° C. and 150 rpm to allow time for complete encapsulation. Following the incubation, the cell cultures were heated in a water bath to 95° C. to kill all uncoated cells. The culture was centrifuged at 4,500×g for 10 minutes to obtain a pellet of spore coated bacterial cells.

### SCN Bacterial Encapsulation

A water bath was heated to 85° C. Next, 100 μL of coated and uncoated cells in microcentrifuge tubes, with three independent replicates for each, were placed in the water for 10 or 75 minutes. The cells were removed from heat and diluted with PBS to 10⁶⁻⁹. The dilutions were plated onto 1.5% agar LB plates and incubated aerobically for 24 hours at 37° C.

100 μL of coated and uncoated cells were added to microcentrifuge tubes, with three independent replicates of each. Next, 1 mL of GFS-Pepsin was added to the tubes and they were incubated for 2 hours at 37° C. and 150 rpm. After incubation, the tubes were centrifuged at 4,500×g to obtain a cell pellet free of GFS. The supernatant was removed and the pellet was resuspended into 1 mL PBS. These cell suspensions were diluted with PBS to 10⁶⁻⁹ then plated onto 1.5% agar LB. Plates were incubated aerobically for 24 hours at 37° C.

100 μL coated and uncoated cells, in triplicate, were added to microcentrifuge tubes and the lids were left open, then all tubes were placed into a 50 mL conical tube and it was covered with a kim wipe. The larger tube was placed into a freeze dry glass container. This was attached to a vacuum and allowed 48 hours for the cells to dry. After two days, the tubes were removed from the vacuum and half were placed into storage at 4° C. for 14 days. The contents of the remaining tubes were resuspended into PBS and diluted to 10⁶⁻⁹. These samples were plated onto 1.5% agar LB and incubated aerobically for 24 hours at 37° C. The three steps above were repeated with the refrigerated samples after 14 days.

On selective media (Xylose Lysine Deoxycholate (XLD) agar for S. enteritidis and MacConkey (MAC) or Tryptone Bile X-Glucuronide (TBX) agar for E. coli), triplicate overnight pathogen cultures were diluted and plated to assess original sample total. On non-selective media, coated cell cultures (that had been heat treated to remove non-coated cells) or uncoated cells, in triplicate, were diluted and plated to assess original sample total. Based on CFU count, the higher concentration sample was diluted to match that of the lower concentration, resulting in ~1×10⁶ cells. In a microcentrifuge tube, 250 μL of the pathogen was added. Next, 250 μL of the coated or uncoated cell was added, based on whether it was the control or experimental culture. An additional 1 mL of fresh LB was added to the culture to allow nutrients for growth. These set ups were run with three independent replicates each. As a total control, one set of tubes with only the pathogen and LB was set up to measure total possible growth. The co-cultures were incubated for 24 hours at 37° C. and 150 rpm. After incubation, the samples were diluted using PBS to 10⁶-12. All samples were plated on both selective media (for the pathogen count) and non-selective media (for the overall cell count). Plates were incubated for 24 hours at 37° C.

### An Illustrative Embodiment

Membrane extrusion is a simple and straightforward method to prepare cell-derived nanomaterials. It can be used to create liposomes and cell membrane-coated nanoparticles. Spore-coat-derived nanomaterials may also be produced by membrane extrusion to coat carrier-free nanoparticles containing therapeutic drugs for colon cancer, enhancing their resistance to the harsh stomach environment.

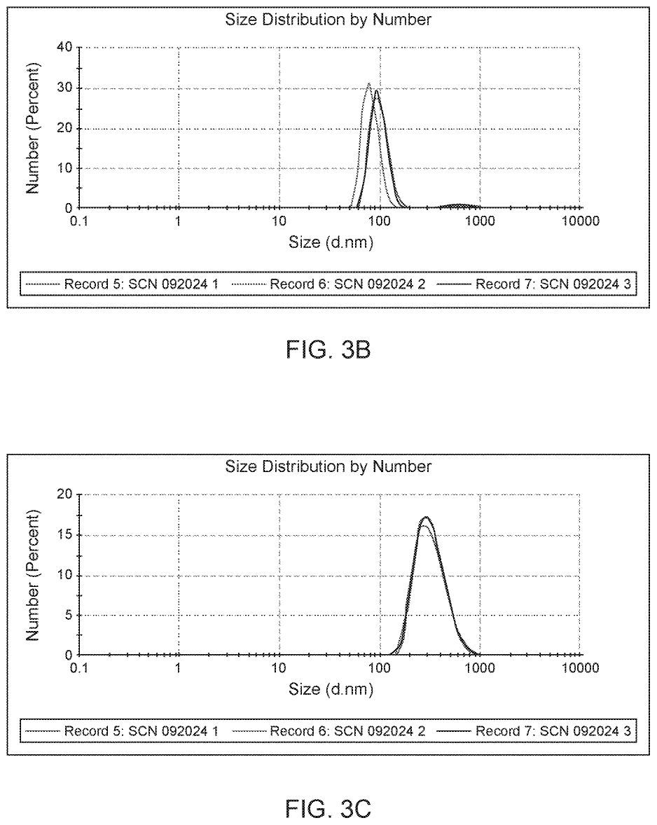

Spore coat nanoparticles (SCN) extracted from B. subtilis 168 M, B. subtilis 6051, and G. stearothermophilus using a series of decreasing filter sizes were produced and purified. Size-based characterization of the SCN shows that SCN developed from G. stearothermophilus are primarily around 100 nm as shown in FIG. 3B, while those produced from Bacillus sp. are larger, around 200 nm as shown in FIG. 3C. Visual inspection using bright-field microscopy and malachite green staining indicates that once purified, almost all of the SCN sample is spore coat material. Visual inspection of SEM images indicate an average SCN size between 100-300 nm as can be seen in FIG. 3A. The size of the SCNs is primarily influenced by the width of the spore coat, which varies significantly among species. In Bacillus subtilis, where the coat is relatively wide, it measures just under 200 nm in width. The size distribution is highly uniform, with a polydispersity index (PDI) of approximately 0.1.

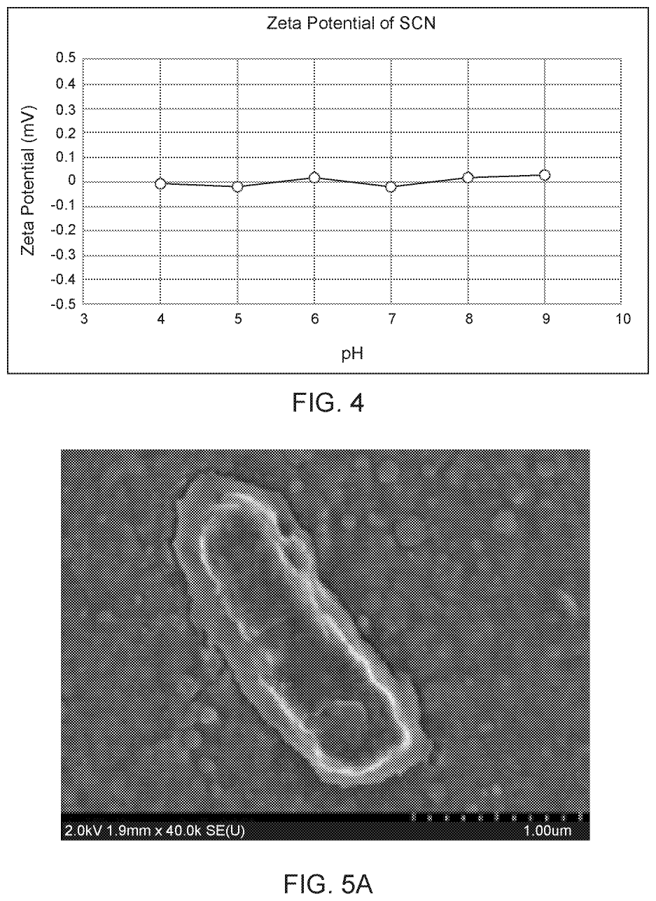

Regardless of the SCN's origin, its zeta potential remains close to zero across a pH range of 4 to 9, as shown in FIG. 4. This behavior is notably different from the reported zeta potential of spores, which is approximately-30 mV at pH 6.5. Other studies have reported values for spore strains at pH 7.0, with wide variations observed between strains, ranging from −12.28 to −44.51 mV. The current spore coat model, based on SEM and AFM analyses, proposes multi-layer structure. The number of layers in the spore coat is species-dependent. In any given spore, the layers typically appear to be in close contact, suggesting they are interconnected by chemical bonds. Partial exposure of each layer on the surface of the SCN may occur during membrane extrusion for SCN preparation.

The Bicinchoninic Acid (BCA) analysis reveals a relatively consistent protein concentration across both species' SCN. Based on a standard curve developed by known protein concentrations between 0.5 μg/mL and 30 μg/mL, nanoparticles produced from both B. subtilis (BS) and G. stearothermophilus (GS) have a protein concentration between 20 and 25 μg/mL, at approximately 23 μg/mL average between the species. These samples are produced from 1 mL of spore suspension that measures 0.15 at OD600, which corresponds to an average of 105 viable spores (N=3). This enumeration suggests that each individual cell produces about 0.2 ng of protein for the spore coat nanoparticles, indicating that spore coat nanoparticles are predominantly made of proteins.

### Cross-Species Coating of SCN

Cross-species SCN encapsulation is the process of using the spore coat material from one species and adhering it to the membrane of a vegetative cell of another species or strain. The goal of cross coating is to determine if SCN encapsulation properties are only provided to cells who have genetic familiarity to the proteins in the coat, and if aggregation of coat protein can only occur on membranes of the cell that produced the coat. In the present state of the art, cross-species coating of SCN has not been performed outside of the Bacillus genus.

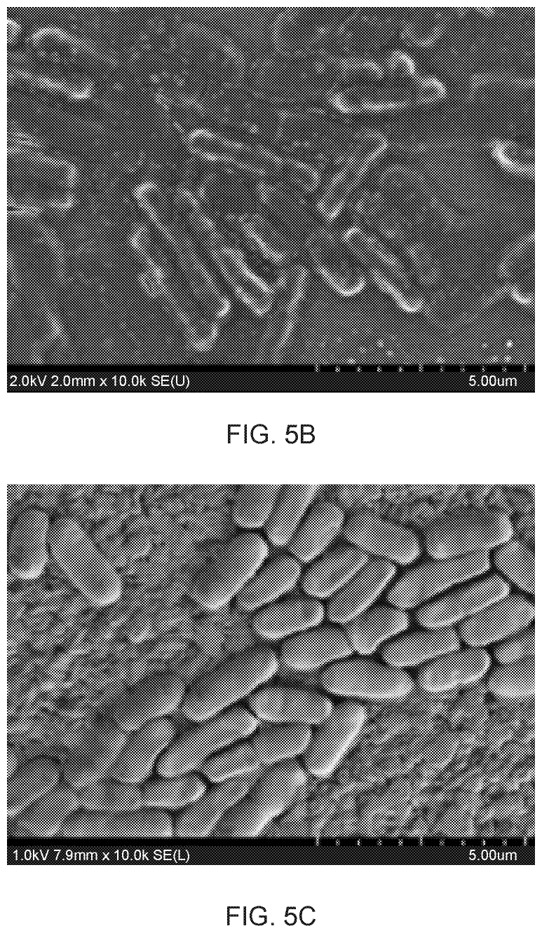

The surfaces of vegetative cells of B. subtilis strains 168M and 6051 coated with SCN were confirmed through SEM analysis, as shown in FIG. 5A. The nanoparticle encapsulation becomes more sparse as cells replicate between 6 and 24 hours of incubation (FIG. 5C). A sparser coverage of nanoparticles may mean lower tolerance of the stressors tested, meaning cells tested after a longer incubation may not be maximized for defense characteristics. However, all resistance properties presented in this illustrated embodiment have been measured after 24 hours of SCN-cell incubation, so it is unknown if a shorter incubation time may lead to more resistance.

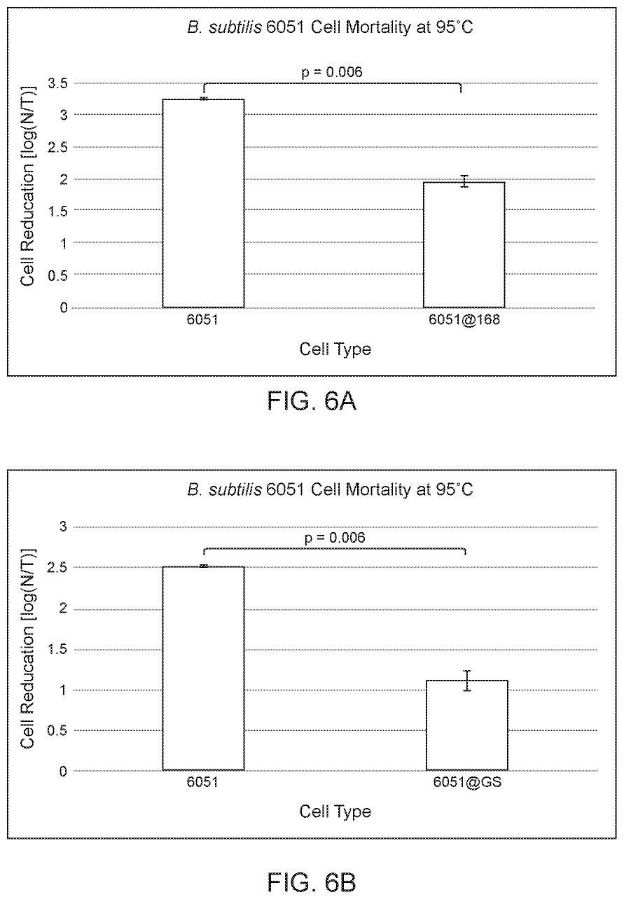

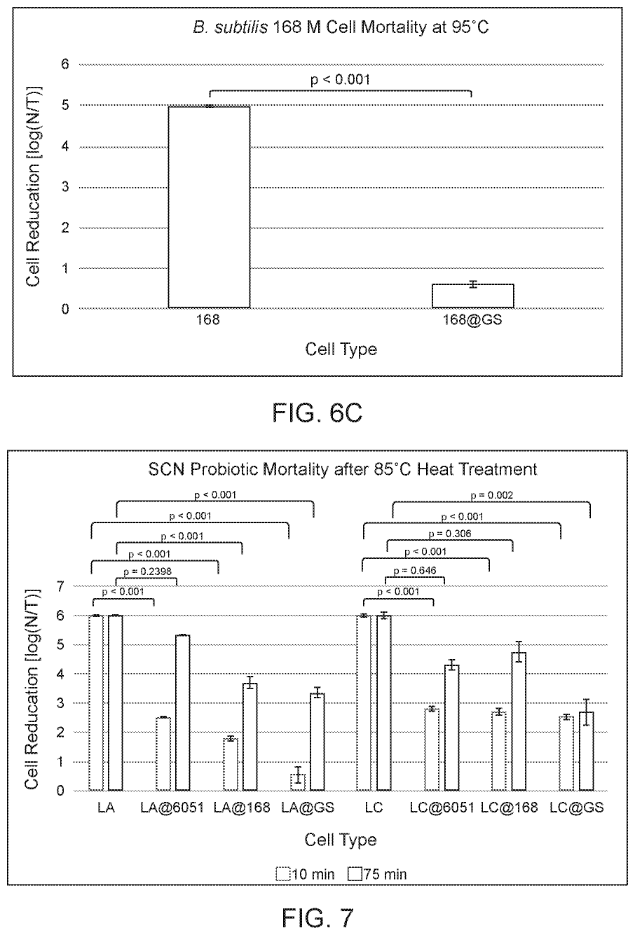

Both B. subtilis strains 168 M and 6051 have increased resistance to heat, acidic pH, and enzymatic digestion when cross-coated with other species' SCN (as from GS). When exposed to 95° C. heat treatments for 10 minutes, coated 168 and 6051 cells survive at rates 2 to 5 log-fold higher than uncoated cells (FIGS. 6A-6C). When treated with gastric fluid simulant with pepsin (GFS-pepsin), at a pH of 1.5-2, for two hours, cross-species coated cells survive 2 log-fold higher than uncoated.

### Improved Survivability of Probiotics In Vitro

The success of cross-species SCN coating on vegetative B. subtilis cells has led to investigating the next step: spore coat experiments for encapsulating non-spore-forming bacteria with proven probiotic characteristics for treating Salmonella sp. and E. coli in poultry. In this illustrated embodiment, Lactobacillus acidophilus (ATCC #BAA-2832) (referred to as LA) and Lactobacillus casei (ATCC #393) (referred to as LC) were selected due to their distant evolutionary relationship from the spore-forming species used to create the SCN, as well as their effectiveness as probiotics in reducing pathogenic bacteria levels in chickens. Following the same protocols for SCN formation and purification described above, the purified spore coat extrusions were incubated at 37° C. and 150 rpm for 24 hours with cultures of the non-spore-forming probiotics LA and LC. After a 24-hour incubation, the cells were centrifuged, resuspended in PBS, and treated at 95° C. for ten minutes to eliminate any uncoated cells from the culture. Staining and SEM image analysis were then performed, as with cross-species coated cells, to verify cell coating. The coated cells were then evaluated for their survivability under diverse stress factors.

Spore coat nanoparticle encapsulation of non-spore-forming probiotics effectively reduces damage from temperature, acidity, enzymatic degradation, desiccation, and long-term storage. During heat treatments at 85° C. for ten minutes, uncoated LA and LC cells showed zero survival, whereas all six cell type and coating combinations demonstrated improved survivability, ranging from 3.5 to 5.5 log (FIG. 7). In heat treatments at 85° C. for 75 minutes, all six combinations survived, with LC@GS showing nearly equivalent survival rates to the ten-minute treatment (FIG. 7). This consistent survival over 10 and 75 minutes suggests LC@GS exhibits enhanced resistance and durability compared to the other combinations.

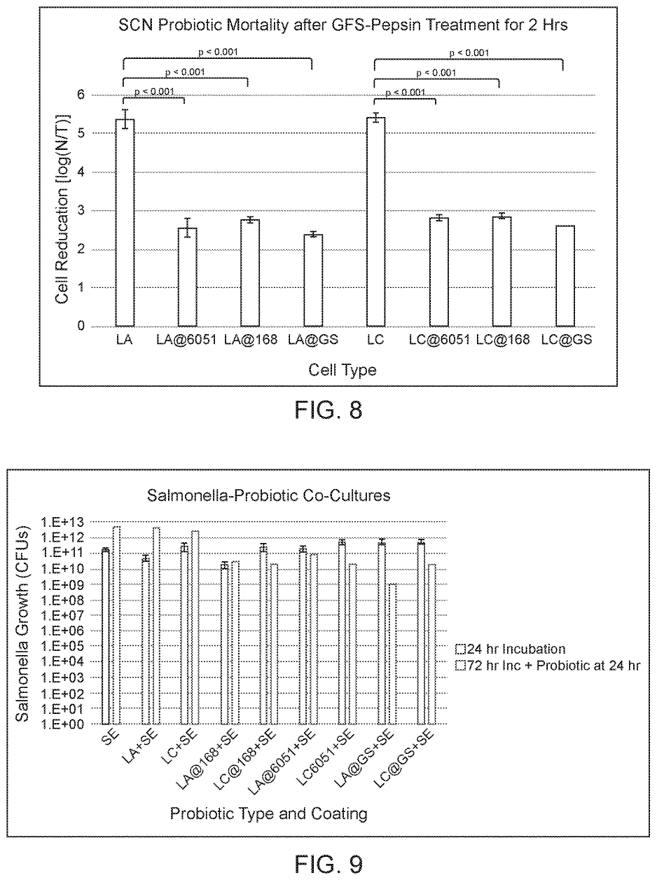

The two-hour aerobic treatment with GFS-pepsin at 37° C. and 150 rpm simulates the mechanical, chemical, and enzymatic digestion conditions encountered during passage through an animal after oral delivery, including shaking, high acidity, and pepsin exposure. Under these conditions, with a pH of approximately 1.5-2, uncoated probiotics show survival of fewer than 10 cells (FIG. 8). In contrast, SCN-encapsulated probiotics exhibit survivability exceeding 3 log for all six cell type and coating combinations.

### Co-Culture Assessment

The next illustrated embodiment determines whether SCN encapsulation interferes with the cells' probiotic capabilities. To evaluate whether spore-coated cells retain their probiotic functions compared to uncoated cells, co-cultures of probiotics with common poultry contaminants and pathogens were conducted to ensure probiotic capacity remains unaffected. Initial experiments examine the differences in total growth of S. enteritidis when cultured alone, with uncoated LC, and with LC@168. Co-cultures were inoculated with equivalent optical densities of the pathogen and the probiotic of interest and incubated at 37° C. and 150 rpm for 24 and 72 hours. After the extended 72-hour incubation with coated probiotics, the Salmonella count was reduced by 2-3 log, as shown in FIG. 9, demonstrating the encapsulated probiotic's ability to inhibit Salmonella growth.

### Experimental Materials Used in Some of the Illustrated Embodiments

Spore coat nanoparticles were produced using a hand-held mini lipid extruder and polycarbonate membranes (Avanti Polar Lipids, #610000-1EA). Spores were suspended in phosphate buffered saline (PBS) pH 7.4 (Invitrogen, #AM9624). Spore suspensions and SCN were stored in 1.5 mL microcentrifuge tubes (Eppendorf, #EP022363531). All cells used, unless specifically stated otherwise, were grown in Luria Broth (Invitrogen, #12795027). Cells were treated with artificial gastric juice with pepsin to simulate a gastrointestinal environment (Carolina Biological, #864603). Lyophilization was carried out using LABCONCO Freeze Dry System 1334. Scanning electron microscopy (SEM) imaging and energy dispersive spectroscopy (EDS) analysis were performed on the Hitachi SU-8030 SEM. The pH of any sample measured was verified using the handheld PH60 Pocket pH Tester (Apera Instruments, #AI311). Zeta size and potential measurements were taken on the Zetasizer Nano ZS. Selective media was used to enumerate pathogen cell growth, on TBX (Millipore Sigma, #1161220500) and MacConkey agar (Millipore Sigma, #70143) for E. coli and XLD (Millipore Sigma, #95586) for S. enteritidis. All BCA and OD readings were collected using a Cary 60 UV-Vis spectrophotometer (Agilent, #G6860A).

The illustrated embodiments drew inspiration from the high resilience of bacterial spores under extreme conditions and have explored using the spore coat as a single-cell encapsulation material, providing protections previously only available to cells capable of forming their own endospores. Bacterial spores are known to withstand various stresses, largely due to the thickness and structure of their multilayered spore coat, composed of over 70 different proteins. Contemporary research demonstrated that lipid-protein nanoparticles derived from spore coats could be extracted and attached to vegetative cells of the same genus, giving these cells advantages over spores. The illustrated embodiments of the invention showed that SCN can also be aggregated onto the surface of non-spore-forming, unrelated, Gram-positive bacteria, providing them with spore-like protection. Moreover, SCN incubation and attachment confer resistance to high thermal extremes and extreme pH-traits previously unknown in these cells. These findings contribute to advancements in biologically-based materials for cellular encapsulation and protection.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

What is claimed is:

1. A method of creating a spore coated probiotic, the method comprising:
preparing a spore suspension;
extruding the spore suspension;
incubating the spore extrusion with a non-spore forming probiotic; and
exposing uncoated cells to a heat treatment to facilitate removal.

2. The method of claim 1, wherein the non-spore probiotic is selected from a group consisting of Lactobacillus acidophilus, Lactobacillus casei, and combinations thereof.

3. The method of claim 1, wherein preparing a spore suspension comprises:
aerobically growing a bacteria;
inoculating a pre-made Spordex Culture Media with the bacteria; and
maturing the cultures.

4. The method of claim 3, wherein the cultures are matured for at least four days.

5. The method of claim 3, wherein the cultures are matured at a temperature of about 4° C.

6. The method of claim 3, wherein the bacteria is selected from a group consisting of Bacillus subtilis 168 M, Bacillus subtilis 6051, Geobacillus stearothermophilus, and combinations thereof.

7. The method of claim 1, wherein the inoculating a pre-made Spordex Culture Media with the bacteria comprises:
incubating the Spordex Culture Media and bacteria for about 24 hours.

8. The method of claim 1, wherein the inoculating a pre-made Spordex Culture Media with the bacteria further comprises:
incubating the Spordex Culture Media and bacteria at 37° C.

9. The method of claim 1, wherein the exposing uncoated cells to a heat treatment comprises:
exposing the uncoated cells for 10 minutes at 85° C.

10. The method of claim 1, wherein the exposing uncoated cells to a heat treatment comprises:
exposing the uncoated cells for 75 minutes at 85° C.
